(19) United States
(12) Patent Application Publication
(10) Pub. No.: US 2025/0041178 A1
(43) Pub. Date: Feb. 6, 2025

(54) SKIN PEELING COMPOSITIONS

(71) Applicant: L'OREAL, Paris (FR)

(72) Inventor: Florence BARRAQUET, CHEVILLY LARUE (FR)

(21) Appl. No.: 18/364,556

(22) Filed: Aug. 3, 2023

(51) Int. Cl. A61K 8/368; A61K 8/365; A61K 8/49; A61Q 19/08

(52) U.S. Cl. CPC A61K 8/368; A61K 8/365; A61K 8/498; A61Q 19/08; A61K 2800/28

(57) ABSTRACT

The present invention relates to compositions including water, monoalcohol, alpha hydroxy acid, beta hydroxy acid, and polyhydroxy acid, and optionally humectant, as well as to methods of using and making such compositions.

### FILED OF THE INVENTION

The present invention relates to compositions comprising water, monoalcohol, alpha hydroxy acids, beta hydroxy acid, and polyhydroxy acid, as well as to methods of using and making such compositions. The compositions have beneficial attribute(s) including, for example, containing solubilized beta hydroxy acid, being well-tolerated during use, effecting peeling at various skin levels, not needing to be neutralized after application to skin, and/or being suitable for use by a consumer at home.

### DISCUSSION OF BACKGROUND

Conventional skin peeling procedures include mechanical removal, e.g., dermabrasion or CO₂ laser, and chemical-induced skin removal. Chemical skin peeling techniques are currently very popular and are often categorized by the degree or amount of skin removal effected.

Chemical peels may be categorized as superficial, medium and deep chemical peels, depending on the depth of chemical wounding of the skin that occurs. Superficial chemical peels are those which remove or effect accelerated replacement or replenishment of the epidermis. Medium depth peels penetrate to the papillary dermis. Deep peels penetrate to the reticular dermis. WO 97/28786 reports that chemical peeling agents include a-hydroxy acids, e.g., glycolic acid or other “fruit acids” such as citric and lactic acids; trichloroacetic acid; phenol, resorcinol and Jessner's solution, an ethanol solution containing equal parts (about 14%) of resorcinol, salicylic acid and lactic acid (85%).

Generally speaking, it is possible to solubilize salicylic acid in some ingredients such as some surfactants and oils. However, the presence of such compounds in a skin peeling composition can have a negative impact on application and feel properties of the composition.

Also, it is possible to solubilize salicylic acid in basic (pH >7) aqueous compositions through formation of a corresponding salicylate. However, in an acidic formulation, salicylic acid tends to precipitate out (not solubilized).

Finally, it is also possible to solubilize smaller amounts of salicylic acid in large amounts of ethanol. However, large amounts of ethanol can have a negative impact on feel and application properties of the composition.

Possibly relevant publications include:

U.S. 2022/0362121 relates to personal care containing an alpha hydroxy acid; a beta hydroxy acid; and polyhydroxy acid, wherein the personal care formulation has a weight ratio of alpha hydroxy acid to polyhydroxy acid is about 5:1 to about 1:4, and wherein the personal care formulation has a weight ratio of beta hydroxy acid to polyhydroxy acid is about 1:1 to about 1:11.

U.S. 2008/0146259 relates to skin peeling compositions containing salicylic acid derivatives.

There remains a need in the art for improved skin peeling compositions, in particular compositions which can be used at home by a consumer, which have good application properties while maintaining salicylic acid solubility.

Accordingly, one aspect of the present invention is a skin peeling composition, in particular a composition which can be used at home by a consumer, which has good properties with respect to composition ease of and/or comfort of application of the composition, as well as solubilized salicylic acid.

### SUMMARY OF THE INVENTION

The present invention relates to skin peeling compositions comprising water, monoalcohol, alpha hydroxy acid, beta hydroxy acid, and polyhydroxy acid.

Preferably, the composition further comprises at least one humectant.

The present invention also relates to skin peeling compositions comprising (a) water, preferably in an amount of 50% or less by weight; (b) 5%-30% by weight of monoalcohol, preferably ethanol, wherein the amount of water present in the composition is greater than the amount of monoalcohol present in the composition, (c) 10%-50% by weight of alpha hydroxy acid, preferably a combination of alpha hydroxy acids including lactic acid, glycolic acid and mandelic acid, (d) 1%-3% by weight of beta hydroxy acid, preferably salicylic acid, and (e) 1%-10% by weight of polyhydroxy acid, preferably gluconolactone, all weights being with respect to the total weight of the composition. Preferably, the composition further comprises at least one humectant in an amount greater than about 5% by weight with respect to the total weight of the composition. Also preferably, the pH of the composition is less than 4.

The present invention also relates to methods of peeling skin comprising applying compositions of the present invention to the skin in an amount sufficient to peel the skin. Preferably, the methods of peeling skin are performed by a consumer and not by a skin care professional using compositions of the present invention.

The present invention also relates to methods of treating skin which contains signs of aging, preferably wrinkles and/or fine lines, comprising applying compositions of the present invention to the skin which contains the signs of aging in an amount sufficient to treat the signs of aging. Preferably, the methods of treating skin which contains signs of aging are performed by a consumer and not by a skin care professional using compositions of the present invention.

The present invention also relates to methods of smoothing skin texture, preferably unevenness of skin resulting from acne, comprising applying compositions of the present invention to the skin in an amount sufficient to make the skin texture more even (smoother). Preferably, the methods of smoothing skin texture are performed by a consumer and not by a skin care professional using compositions of the present invention.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Substituted” as used herein, means comprising at least one substituent.

Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Physiologically acceptable” means compatible with skin and having an acceptable pleasant color, odor and feel, and which does not cause any unacceptable discomfort liable to discourage a consumer from using the composition. Acceptable pH levels for compositions of the present invention are acidic, that is, less than 7, preferably 6 or less, preferably 5 or less, and preferably 4 or less, including all ranges and subranges therebetween such as, for example 3 to 5, 4 to 6, 2 to 4, 3 to 4, etc.

“Natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

The compositions and methods of the present invention can comprise, consist of, or consist essentially of the essential elements and limitations of the invention described herein, as well as any additional or optional ingredients, components, or limitations described herein or otherwise useful. For example, the solvent system can “consist essentially of” water and C2-C4 monoalcohol, or “consist essentially of water, C2-C4 monoalcohol, and humectant such as a glycol or polyol.”

For purposes of the present invention, the “basic and novel property” associated with compositions, components and methods which “consist essentially of” identified ingredients or actions is “beta hydroxy acid (BHA) solubility.”

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of botanical extracts” means that an effective amount (that is, more than trace amounts) of botanical extracts is omitted from the composition (that is, about 0% by weight), “substantially free of botanical extracts” means that botanical extracts is/are present in amounts not greater than 0.1% by weight, and “devoid of botanical extracts” means that botanical extracts is/are present in amounts not greater than 0.25% by weight, based on the total weight of the composition. “Botanical extract” includes witch hazel, and the compositions can also be “free of,” “substantially free of” and “devoid of” witch hazel.

The same nomenclature applies for all other ingredients identified throughout the application and in the preceding paragraph such as, for example, oils and/or waxes, and surfactants (compositions of the invention which are “free of oils and/or waxes,” “substantially free of oils and/or waxes,” and “devoid of oils and/or waxes,” and/or “free of surfactants,” “substantially free of surfactants,” and “devoid of surfactants” have meanings consistent with the discussion within the preceding paragraph), even if not specifically discussed for each identified ingredient.

Discussed examples of the use of such language such as those in this and the preceding paragraph are intended to be exemplary embodiments of the present invention, not limiting.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Alpha Hydroxy Acids (AHAs)

According to the present invention, compositions comprising at least one AHA are provided. AHAs are known in the art, representative examples of which include citric acid, gluconic acid, glycolic acid, lactic acid, malic acid, mandelic acid, tartaric acid, etc.

According to preferred embodiments, compositions of the present invention include more than one AHA. Preferably, compositions of the present invention containing more than one AHA include mandelic acid. More preferred compositions contain glycolic acid and lactic acid in addition to mandelic acid.

Preferably, the at least one AHA is present in the compositions of the present invention in a total (combined) amount ranging from about 7.5% to about 60% by weight, preferably from about 10% to about 50% by weight, preferably from about 15% to about 40%, and preferably from about 20% to about 30% by weight with respect to the weight of the composition, including all ranges and subranges therebetween such as, for example, 11% to 40%, 20% to 25%, 15% to 25%, etc.

Preferably, it mandelic acid is present, it is present in the compositions of the present invention in an amount ranging from about 0.5% to about 25% by weight, preferably from about 1% to about 20% by weight, preferably from about 1% to about 10%, and preferably from about 1% to about 5% by weight with respect to the weight of the composition, including all ranges and subranges therebetween such as, for example, 11% to 20%, 20% to 25%, 15% to 25%, 1% to 3%, etc.

Preferably, if glycolic acid is present, it is present in the compositions of the present invention in an amount ranging from about 2.5% to about 20% by weight, preferably from about 5% to about 15% by weight, preferably from about 6% to about 12.5%, and preferably from about 7% to about 10% by weight with respect to the weight of the composition, including all ranges and subranges therebetween such as, for example, 11% to 20%, 10% to 15%, 12.5% to 15%, 5% to 10%, etc.

Preferably, if lactic acid is present, it is present in the compositions of the present invention in an amount ranging from about 2.5% to about 20% by weight, preferably from about 5% to about 15% by weight, preferably from about 6% to about 12.5%, and preferably from about 7% to about 10% by weight with respect to the weight of the composition, including all ranges and subranges therebetween such as, for example, 11% to 20%, 10% to 15%, 12.5% to 15%, 5% to 10%, etc.

### Beta Hydroxy Acids (BHAs)

According to the present invention, compositions comprising at least one BHA are provided. BHAs are known in the art, representative examples of which include salicylic acid, beta hydroxybutanoic acid, tropic acid, and trethocanic acid. Preferably, compositions of the present invention comprise salicylic acid.

Preferably, the at least one BHA is present in the compositions of the present invention in an amount ranging from about 0.5% to about 5% by weight, preferably from about 1% to about 3% by weight, preferably from more than (or greater than) 1% to about 3%, and preferably from more than 1% to about 2% by weight with respect to the weight of the composition, including all ranges and subranges therebetween such as, for example, 1.25% to 2%, 1.3% to 2%, 1.5% to 2%, etc.

According to preferred embodiments, the AHA(s) and the BHA(s) are present in the compositions of the present invention in a weight ratio of about 15:1 to about 3:1, preferably about 12:1 to about 5:1, and preferably about 11:1 to about 7:1, including all ranges and subranges therebetween.

According to preferred embodiments, where salicylic acid and mandelic acid are present in the compositions of the present invention, the salicylic acid and mandelic acid are present in the compositions of the present invention in a weight ratio of about 1:10 to about 4:1, preferably about 1:5 to about 3:1, and preferably about 1:1 to about 2:1, including all ranges and subranges therebetween. According to some such preferred embodiments, more salicylic acid is present in the compositions than mandelic acid.

### Polyhydroxy Acids (PHAs)

According to the present invention, compositions comprising at least one PHA are provided. PHAs are known in the art, representative examples of which include gluconolactone, galactose and lactobionic acid. Preferably, compositions of the present invention comprise gluconolactone.

Preferably, the at least one PHA is present in the compositions of the present invention in an amount ranging from about 0.5% to about 25% by weight, preferably from about 1% to about 20% by weight, preferably from about 1% to about 10%, and preferably from about 5% to about 10% by weight with respect to the weight of the composition, including all ranges and subranges therebetween such as, for example, 4% to 14%, 1% to 15%, 5% to 25%, etc.

According to preferred embodiments, the AHA(s) and the PHA(s) are present in the compositions of the present invention in a weight ratio of about 25:1 to about 1:1, preferably about 12:1 to about 2:1, and preferably about 6:1 to about 2.5:1, including all ranges and subranges therebetween.

According to preferred embodiments, the PHA(s) and the BHA(s) are present in the compositions of the present invention in a weight ratio of about 15:1 to about 1:1, preferably about 10:1 to about 2:1, and preferably about 6:1 to about 2.5:1, including all ranges and subranges therebetween.

According to preferred embodiments, where gluconolactone and mandelic acid are present in the compositions of the present invention, the gluconolactone and mandelic acid are present in the compositions of the present invention in a weight ratio of about 1:10 to about 10:1, preferably about 1:5 to about 5:1, and preferably about 5:1 to about 2:1, including all ranges and subranges therebetween. According to some such preferred embodiments, more gluconolactone is present in the compositions than mandelic acid.

### Water/Monoalcohol

According to the present invention, compositions comprising an aqueous phase solvent system comprising (i) water in an amount of at least about 5% by weight with respect to the total weight of the composition; and (ii) at least one C2-C4 monoalcohol are provided.

According to the present invention, the compositions comprise water. Preferably, the compositions comprise from about 10% to about 50% water by weight, preferably from about 15% to about 50%, preferably from about 20% to less than 50%, preferably from about 25% to about 45%, and preferably from about 30% to about 45%, by weight of water, all weights being based on the total weight of the composition, including all ranges and subranges therebetween such as, for example, about 25% to less than 50%.

According to the present invention, the compositions comprise at least one monoalcohol. Suitable C2-C4 monoalcohols include, for example, ethanol, propanol, butanol, isopropanol, and isobutanol. Ethanol is particularly preferred.

Preferably, the C2-C4 monoalcohol(s) is/are present in the compositions of the present invention in an amount ranging from about 1% to about 30% by weight, preferably from about 2.5% to about 30% by weight, preferably from about 5% to about 30% by weight, and preferably from about 10% to about 25%, by weight, all weights being based on the total weight of the composition, including all ranges and subranges in between such as, for example, about 10 to about 30%.

Preferably, more water is present in the compositions of the present invention than C2-C4 monoalcohol.

Preferably, the aqueous phase solvent system of the compositions of the present invention consists essentially of, or consists of, water and C2-C4 monoalcohol(s). Preferably, the aqueous phase solvent system is “free of,” “devoid of” or “substantially free of” solvents other than water and C2-C4 monoalcohol(s).

### Humectant

According to preferred embodiments, compositions of the present invention optionally further comprise at least one humectant. Preferably, the at least one humectant comprises at least one hydroxy group or is a derivative of a compound comprising at least one hydroxy group. Suitable humectants of this type are known in the art and include, but are not limited to, propylene glycol, butylene glycol, hexylene glycol, glycerine (sometimes known as glycerol or glycerin) and glucose ethers. Preferably, the at least one humectant is selected from glycerine, propylene glycol, butylene glycol, and mixtures thereof.

If present, the at least one humectant is present in the compositions of the present invention in an amount ranging from about 0.5% to about 25% by weight, preferably from about 1% to about 20% by weight, preferably from about 2.5% to about 15%, and preferably from about 5% to about 10% by weight with respect to the weight of the composition, including all ranges and subranges therebetween such as, for example, 1% to 10%, 5% to 15%, 15% to 25%, 1% to 5%, etc.

However, according to some embodiments, compositions of the present invention are “free of,” substantially free of,” or “devoid of” humectant.

### Additional Optional Ingredients

The composition of the invention can also comprise any additive usually used in the field under consideration. For example, film forming agents, gelling agents, surfactants, oils, waxes, botanical extracts, dispersants, preserving agents, fragrances, fillers, antioxidants, neutralizing agents, silicone elastomers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9th ed. 2002). However, it is to be understood that preferred embodiments of the present invention include compositions which are “free,” substantially free” or “devoid” of the ingredients discussed in this paragraph such as oils, waxes, surfactants, botanical extracts, etc.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

Needless to say, the composition of the invention should be cosmetically or dermatologically acceptable, i.e., it should contain a non-toxic physiologically acceptable medium.

The skin peeling compositions of the present invention may be applied in any manner, for example, by wiping, dispensing, etc. (hereinafter “applying”). For example, applying the invention compositions may be accomplished manually such as, for example, using one or more fingers, using an absorbent cotton swab wetted with the concentrated solution, using a solution-wetted sable brush, gentle wiping with a solution-wetted absorbent fibrous material such as a gauze square or nonwoven pad, etc. Preferably, (1) the compositions of the present invention are applied by a consumer, not a skin care professional, preferably in the consumer's home; (2) the compositions of the present invention are applied using one or more fingers; and/or (3) areas such as eyes and lips are avoided during application of the skin peeling composition of the present invention.

The applied compound or composition is typically allowed to air dry over a relatively short period of time, preferably 20 minutes or less, and preferably 15 minutes or less, with approximately 15 minutes being preferred. A single uniform application of the composition to the skin to be treated and/or its surroundings is generally sufficient.

Once the applied composition has been on the skin (e.g., the face) for sufficient time, the composition can be removed preferably by washing or rinsing with water. Preferably, the treated skin is washed or wiped with water, e.g., with a water-moistened or water-wet swab, gauze square, or the like.

Compositions of the present invention can be removed by methods consisting only of rinsing or washing such as that described above. No separate neutralization step or composition prior to removal is necessary for the skin peel compositions of the present invention.

The application process can be repeated periodically as needed, for example application can be repeated about every 2 to 7 days, preferably every 2 to 5 days, preferably every 2 to 3 days, with repeated application every other day being preferred.

Prior to application of the compositions of the present invention, skin is preferably cleaned, but such cleaning is optional.

According to preferred embodiments of the present invention, methods of peeling skin comprising applying compositions of the present invention to the skin in an amount sufficient to peel the skin are provided. Preferably, the methods of peeling skin are performed by a consumer and not by a skin care professional using compositions of the present invention as discussed above.

According to preferred embodiments of the present invention, methods of treating skin which contains signs of aging, preferably wrinkles and/or fine lines, comprising applying compositions of the present invention to the skin which contains the signs of aging in an amount sufficient to reduce the appearance of the fine lines and/or wrinkles are provided. Preferably, these methods are performed by a consumer and not by a skin care professional using compositions of the present invention as discussed above.

According to preferred embodiments of the present invention, methods of smoothing skin texture, preferably unevenness of skin resulting from acne, comprising applying compositions of the present invention to the skin in an amount sufficient to make the skin texture more even (smoother) are provided. Preferably, these methods are performed by a consumer and not by a skin care professional using compositions of the present invention as discussed above.

According to preferred embodiments of the present invention, methods of making a skin peeling composition are provided which comprise:
(1) preparing a basic aqueous medium by adding at least one basifying agent to water, preferably water having a temperature at or below room temperature, in an amount sufficient to produce a basic (pH above 7) aqueous medium,
(2) adding AHA(s) and PHA(s) one by one to the basic aqueous medium of (1) to gradually reach the approximate final pH of the skin peeling composition,
(3) adding the remainder of ingredients of the skin peeling composition (except BHA(s), monoalcohol(s) and, optionally, humectant(s)) to the aqueous medium of (2), and
(4) adding a mixture of BHA(s), monoalcohol(s) and, optionally, humectant(s) to the aqueous medium of (3).

Any basifying agent known in the art can be used in accordance with these methods. Suitable basifying agents include, but are not limited to, at least one basifying agent selected from the group consisting of alkanolamines such as mono-, di- and triethanolamines, alkali metal carbonate salts, sodium hydroxide, potassium hydroxide or calcium hydroxide, and mixtures thereof, with sodium hydroxide being most preferred.

Any monoalcohol known in the art can be used in accordance with these methods. Suitable C2-C4 monoalcohols include, for example, ethanol, propanol, butanol, isopropanol, and isobutanol. Ethanol is particularly preferred.

Any humectant known in the art can be used in accordance with these methods. Preferably, the at least one humectant comprises at least one hydroxy group or is a derivative of a compound comprising at least one hydroxy group.

Suitable humectants of this type are known in the art and include, but are not limited to, propylene glycol, butylene glycol, hexylene glycol, glycerine (sometimes known as glycerol or glycerin) and glucose ethers. Preferably, the at least one humectant is selected from glycerine, propylene glycol butylene glycol, and mixtures thereof.

Preferably, in (4), BHA(s) such as salicylic acid is/are solubilized in the monoalcohol and, optionally, humectant prior to addition to the aqueous medium of (3).

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Preparation of Sample Composition

A sample composition set forth in the table below can be prepared as follows:

| | |
|---|---|
| WATER/AQUA | 10-50% |
| SODIUM HYDROXIDE | 1-10% |
| CHELATING AGENT | 0.01-0.1% |
| GLUCONOLACTONE | 1-10% |
| MANDELIC ACID | 0.5-25% |
| LACTIC ACID | 2.5-20% |
| GLYCOLIC ACID | 2.5%-20% |
| COLORANT | 0-5% |
| GELLING AGENT | 0-5% |
| ALCOHOL DENAT. | 5-30% |
| SALICYLIC ACID | >1% to 3% |
| OTHER INGREDIENTS SUCH AS | QS |
| HUMECTANT, COOLING AGENT, | |
| FRAGRANCE, | |

Introduce sodium hydroxide (NaOH) into the water in a cold tank to produce a basic medium.

AHA and PHA are added one by one in the basic medium to gradually reach approximately the final pH of the formula.

The remainder of ingredients except salicylic acid, ethanol, and (if present) hydroxy-containing humectant are added after AHA and PHA addition.

Salicylic acid is solubilized in a phase of denatured alcohol (e.g., ethanol) and (if present) hydroxy-containing humectant (e.g., glycerol), and then added to the rest of the formula.

This procedure can make it possible to prepare this composition without crystalized salicylic acid, and the composition can be stable for up to 2 months (no recrystallization after 2 months at 4° C.).

### Example 2—Invention and Comparative Compositions

The compositions below were prepared according to the description in example 1:

| Ingredient | Invention A | Comparative B | Comparative C |
|---|---|---|---|
| WATER | QS | QS | QS |
| LACTIC ACID | 10.00 | 10.00 | 10.00 |
| (AHA) | | | |
| GLYCOLIC | 7.00 | 7.00 | 7.00 |
| ACID (AHA) | | | |
| MANDELIC | 1.00 | 1.00 | 1.00 |
| ACID (AHA) | | | |
| GLUCONO- | 5.00 | 5.00 | 5.00 |
| LACTONE (PHA) | | | |
| SALICYLIC | 2.00 | 1.00 | 0.50 |
| ACID (BHA) | | | |
| GLYCERIN | 8.00 | 8.00 | 8.00 |
| (GLYCOLS) | | | |
| ALCOHOL DENAT. | 5.00 | 5.00 | 5.00 |
| POLYACRYLATE | 0.90 | 0.90 | 0.90 |
| CROSSPOLYMER- | | | |
| 6 (GELLING AGENT) | | | |
| SODIUM | 4.00 | 4.00 | 4.00 |
| HYDROXIDE | | | |
| OTHERS (Actives | 0.40 | 0.40 | 0.40 |
| ingredients, perfume, | | | |
| CHELATING AGENT) | | | |

### Example 3—Testing for Salicylic Acid Solubility

At the end of preparation of the compositions from Example 2, the microscopic appearance of the compositions was examined. It could be seen that Invention Example A (2% salicylic acid) did not contain visible crystals. However, comparative examples containing 1%0 or less of salicylic acid (Comparative Example B (1% salicylic acid) and Comparative Example C (0.5% salicylic acid)) contained visible crystals, with Comparative Example C containing more crystals than Comparative Example B. These results were surprising and could not have been expected given that more crystal formation occurred in compostions containing less salicylic acid. It was determined that more than 1% salicylic acid should be present to obtain acceptable salicylic acid solubility.

1. A skin peeling composition comprising:
(a) about 50% by weight or less of water,
(b) about 5% to about 30% by weight of monoalcohol(s), wherein the amount of water present in the composition is greater than the amount of monoalcohol(s) present in the composition,
(c) about 10% to about 50% by weight of alpha hydroxy acid(s),
(d) more than 1% to about 3% by weight of salicylic acid, and
(e) about 1% to about 10% by weight of polyhydroxy acid(s),
all weights being with respect to the total weight of the composition.

2. The composition of claim 1, wherein the salicylic acid is present in an amount of more than 1% to about 2% by weight with respect to the total weight of the composition, preferably from about 1.5% to about 2% by weight with respect to the total weight of the composition.

3. The composition of claim 1, wherein one monoalcohol present in the composition is ethanol.

4. The composition of claim 1, wherein one alpha hydroxy acid present in the composition is mandelic acid, preferably in an amount ranging from about 1% to about 10% by weight with respect to the total weight of the composition, preferably from about 1% to about 5% by weight with respect to the total weight of the composition.

5. The composition of claim 4, wherein two additional alpha hydroxy acids present in the composition are lactic acid and glycolic acid.

6. The composition of claim 1, wherein one poly hydroxy acid present in the composition is gluconolactone, preferably in an amount ranging from about 1% to about 10% by weight with respect to the total weight of the composition, preferably from about 5% to about 10% by weight with respect to the total weight of the composition.

7. The composition of claim 1, further comprising at least one humectant, preferably in an amount greater than 5% by weight with respect to the total weight of the composition.

8. The composition of claim 1, wherein the pH of the composition is 4 or less.

9. A method of peeling skin comprising applying the skin peeling composition of claim 1 to skin in an amount sufficient to peel the skin, and rinsing the composition off the skin with water after peeling.

10. A method of smoothing skin texture comprising applying the skin peeling composition of claim 1 to skin in an amount sufficient to make the skin texture smoother.

11. The composition of claim 1, comprising:
(a) about 50% by weight or less of water,
(b) about 5% to about 30% by weight of monoalcohol(s), wherein the amount of water present in the composition is greater than the amount of monoalcohol(s) present in the composition,
(c) about 15% to about 20% by weight of alpha hydroxy acid(s),
(d) about 2% by weight of salicylic acid, and
(e) about 5% to about 10% by weight of polyhydroxy acid(s).

12. A method of making a skin peeling composition comprising salicylic acid, at least one monoalcohol and optionally at least one humectant, comprising:
(a) preparing a basic aqueous medium by adding at least one basifying agent to water in an amount sufficient to produce a basic aqueous medium,
(b) adding alpha hydroxy acid(s) and polyhydroxy acid(s) one by one to the basic aqueous medium to reach an approximate final pH of the skin peeling composition,
(c) adding remaining ingredients of the composition except the salicylic acid, at least one monoalcohol, and optionally at least one humectant, to the aqueous medium having the approximate final pH of the skin peeling composition, and
(d) adding a mixture of the salicylic acid, at least one monoalcohol, and optionally at least one humectant to the aqueous medium of (3) to prepare the skin peeling composition.
